(19) United States
(12) Patent Application Publication
(10) Pub. No.: US 2008/0119519 A1
(43) Pub. Date: May 22, 2008

(54) Base Forming Drug-Layered Silicate Hybrid Containing Basic Polymer and its Synthesis Method

(75) Inventors: Jin-Ho Choy, Seoul (KR); Taeun Park, Seoul (KR); Yoon-joo Lee, Seoul (KR); Dong-Youn Kim, Kyungki-do (KR); Jin-Kuen Park, Seoul (KR)

Correspondence Address: ROTHWELL, FIGG, ERNST & MANBECK, P.C., 1425 K STREET, N.W., SUITE 800, WASHINGTON, DC, 20005, US

(73) Assignee: NANOHYBRID CO., LTD., Seoul (KR)

(21) Appl. No.: 11/816,756

(22) PCT Filed: Feb. 21, 2006
(86) PCT No.: PCT/KR06/00579

§ 371 Date: Aug. 21, 2007

(30) Foreign Application Priority Data
Feb. 21, 2005 (KR) 10-2005-0014273

(51) Int. Cl. A61K 47/48; A61K 31/4525; A61K 31/451

(52) U.S. Cl. 514/321; 514/317; 514/770

(57) ABSTRACT

Provided are a hybrid of a poorly soluble basic drug and a layered silicate, including a water-soluble basic polymer, and a method of preparing the same. The water-soluble basic polymer may be an aminoalkylmethacrylate copolymer (e.g., Eudragit E 100: a copolymer composed of 1:2:1 ratio of butyl methacrylate, (2,2-dimethylaminoethyl)methacrylate, and methyl methacrylate, Degussa) or polyvinylacetal diethylaminoacetate (AEA). In an oral formulation including the hybrid, a short-term dissolution of the poorly soluble drug can be increased to 90%, thereby increasing drug bioavailability.

### CROSS-REFERENCE TO RELATED PATENT APPLICATION(S)

This application is a 35 U.S.C. § 371 National Phase Entry Application from PCT/KR2006/000579, filed Feb. 21, 2006, and designating the United States. This application also claims the benefit of Korean Patent Application No. 10-2005-0014273, filed on Feb. 21, 2005, in the Korean Intellectual Property Office, the disclosure of which is incorporated herein in its entirety by reference.

### BACKGROUND OF THE INVENTION

1. Field of the Invention

The present invention relates to a method of preparing a hybrid of a poorly soluble basic drug and a layered silicate to solubilize the drug, a hybrid composition including a basic organic material to achieve a pharmaceutically desired dissolution, and a method of preparing the composition.

2. Description of the Related Art

Generally, poorly soluble drugs have very low solubility in water, and thus, their dissolutions within a predetermined time after oral administration are also low, thereby decreasing the in vivo absorption of the drugs. In order to make poorly soluble drugs into oral dosage forms, novel processes have been developed to prepare amorphous forms of the poorly soluble drugs. However, the amorphous forms may give rise to the problem of drug instability. Thus, new attempts have been made to overcome this problem. Its most representative example is a method of preparing a poorly soluble drug in the form of a pharmaceutically acceptable anion-containing non-toxic acid addition salt or a crystalline free base.

By way of another example, a poorly soluble drug can be made into its hybrid with a layered silicate that is a layered inorganic carrier. In particular, Japanese Patent Application No. 2000-88403 discloses the preparation of a hybrid composite of a layered silicate with each of indometacin and bufexamac that are anti-inflammatory drugs. These hybrid composites, used as skin ointments, show good drug efficacy and low irritation, but have a serious limitation for use as oral formulations due to low dissolution.

Meanwhile, in order to increase the bioavailability and stability of amlodipine that is a poorly soluble drug for the treatment of hypertension, the present inventors developed a hybrid of a free-base form of amlodipine and a layered silicate, and verified an enhancement in dissolution of amlodipine (Korean Patent Application No. 10-2003-0057890).

Therefore, while searching for a solution to the above problems related to the prior art, the present inventors found that when a basic polymer was added to a hybrid of a free-base form of a drug and a layered silicate, the dissolution of the drug was remarkably increased, and thus, completed the present invention.

### SUMMARY OF THE INVENTION

The present invention provides a basic polymer-containing hybrid of a layered silicate and a free-base form of a drug, which shows high dissolution characteristics suitable for use as oral dosage forms, and a method of preparing the hybrid. Therefore, a reduction in dissolution that may be caused when a poorly soluble basic drug, and an inorganic carrier, such as a layered silicate, which has been developed to increase the bioavailability and stability of the poorly soluble basic drug, are formulated into oral dosage forms, can be solved.

As described above, the present inventors have already developed a method of loading an amlodipine free base onto an inorganic carrier in a solvent, such as ethanol and distilled water, to effectively release amlodipine. The present inventors have also already developed an amlodipine-carrier hybrid capable of solubilizing a poorly soluble amlodipine free base using a hybrid technique for hybrid synthesis of a drug and an inorganic carrier.

According to an aspect of the present invention, there is provided a hybrid of a drug in a free-base form and a layered silicate, including a basic polymer capable of controlling an dissolution of the drug, wherein the drug is inserted into an interlayer of the layered silicate. According to the present invention, when a cationic polymer is added to a drug-inorganic carrier hybrid with a low drug dissolution, the dissolution of the drug can be increased to about 90%.

The layered silicate is not particularly limited provided that it has a layered structure and its interlayer space contains an alkaline metal ion or an alkaline earth metal ion. The layered silicate may be selected from the group consisting of montmorillonite, beidellite, nontronite, hectorite, saponite, illite, celadonite, gluconite, clay, and bentonite.

The drug in the free-base form may be any drug that has high basicity and thus can be substituted for the alkaline metal ion or the alkaline earth metal ion in the interlayer space of the layered silicate. The drug may be selected from the group consisting of amlodipine, paroxetine, donepezile, and sibutramin.

The basic polymer can be added alone to the hybrid of the drug and the layered silicate, but may be added in combination with an inorganic salt. Here, the inorganic salt may be selected from the group consisting of a calcium salt, a sodium salt, a potassium salt, and an ammonium salt.

The weight ratio of the basic polymer to the drug may be 0.3-30, and more preferably 0.5-1.0. If the content of the basic polymer is too small, it may be difficult to achieve an ideal drug dissolution suitable for oral formulations. On the other hand, if it is too much, the efficacy of the drug may be adversely affected.

The basic polymer may be any water-soluble polymer with a pharmaceutically acceptable cation, and preferably, a cationic polymer or copolymer.

In particular, the basic polymer may be an alkylamino methacrylate copolymer (e.g., Eudragit E 100; a copolymer composed of 1:2:1 ratio of butyl methacrylate, (2,2-dimethylaminoethyl)methacrylate, and methyl methacrylate, Degussa), polyvinylacetal diethylaminoacetate (AEA), or polyalkylaminoalkylmethacrylate.

According to another aspect of the present invention, there is provided a method of preparing the hybrid of the present invention, the method including: (a) dispersing a layered silicate in an aqueous solvent to prepare a layered silicate-containing aqueous solution; (b) dissolving a drug in a free-base form in ethanol or water to prepare a drug-containing solution; (c) mixing the layered silicate-containing aqueous solution with the drug-containing solution while stirring to prepare a hybrid wherein the drug is inserted into an interlayer of the layered silicate; and (d) adding a basic polymer to the hybrid.

In (b), the ethanol or the water may have pH of 1-7. This is because a reaction between the drug with high basicity and the layered silicate efficiently occurs in an acidic condition of pH 1-7.

In (d), the basic polymer may be added to the hybrid using any method known in the art, preferably, spray drying, fluidized-bed coating, vacuum drying, or a common oven drying.

According to still another aspect of the present invention, there is provided an oral formulation including the hybrid of the present invention as an effective ingredient

### BRIEF DESCRIPTION OF THE DRAWINGS

The above and other features and advantages of the present invention will become more apparent by describing in detail exemplary embodiments thereof with reference to the attached drawings in which:

FIG. 1 is an X-ray diffraction pattern of a paroxetine-montmorillonite hybrid;

FIG. 2 is a graph illustrating the results for an dissolution test with a pH 1.2 buffer for a paroxetine-montmorillonite hybrid and an Eudragit E-coated paroxetine-montmorillonite hybrid;

FIG. 3 is a graph illustrating a paroxetine dissolution of a Eudragit E-coated paroxetine-montmorillonite hybrid with respect to an addition amount of Eudragit E;

FIG. 4 is an X-ray diffraction pattern of a donepezile-montmorillonite hybrid;

FIG. 5 is a graph illustrating the results for an dissolution test with a pH 1.2 buffer for a donepezile-montmorillonite hybrid and an Eudragit E-coated donepezile-montmorillonite hybrid;

FIG. 6 is an X-ray diffraction pattern of a sibutramin-montmorillonite hybrid; and

FIG. 7 is a graph illustrating the results for an dissolution test with a pH 1.2 buffer for a sibutramin-montmorillonite hybrid, an Eudragit E-coated sibutramin-montmorillonite hybrid, and an AEA-coated sibutramin-montmorillonite hybrid.

### DETAILED DESCRIPTION OF THE INVENTION

The present invention will now be described more fully with reference to the accompanying drawings, in which exemplary embodiments of the invention are shown.

In a hybrid of a drug in a free-base form and a layered silicate (hereinafter, also referred to simply as “drug-layered silicate hybrid”) according to the present invention, the layered silicate is used as a carrier for the drug in the free-base form. For example, the layered silicate may be montmorillonite, beidellite, nontronite, hectorite, saponite, illite, celadonite, gluconite, clay, or bentonite.

The layered silicate has a layered structure and its interlayer space contains an alkaline metal ion or an alkaline earth metal ion. These ion species can be easily substituted by cationic organic materials (drugs), and thus, the layered silicate is used for stabilization and supporting of the organic materials.

Silicates have a pyramidal SiO4 tetrahedron as a building block. Layered silicates have a unit layer structure with a metal cation (e.g., aluminum) sandwiched between two sheets of SiO4 tetrahedra (e.g., Si—Al—Si unit layer structure). Here, the SiO4 tetrahedra are arranged so that vertex oxygen atoms of the SiO4 tetrahedra of one sheet face with those of the other sheet, and a vertex oxygen atom of each SiO4 tetrahedron is bound to the metal cation. The vertical arrangement of the unit layer structure forms a layered structure.

Since a silicon atom of the SiO4 tetrahedron, which is a fundamental building block of the layered structure, can be substituted by a metal cation (e.g., aluminum), the layered structure is wholly negatively charged, and thus, has charge exchange capacity. In order to compensate for negative charges, alkaline metal cations or alkaline earth metal cations (e.g., Na+, Ca2+) exist in the interlayers of the layered structure.

Preferable layered silicates that can be used herein are montmorillonite, beidellite, hectorite, saponite, and illite, which can be respectively represented by Formulae 2-6 below. Formulae 2-6 merely represent general chemical compositions of available montmorillonite, beidellite, hectorite, saponite, and illite. Thus, the chemical compositions of available montmorillonite, beidellite, hectorite, saponite, and illite are not limited by Formulae 2 through 6, and may vary slightly.

(Al₂₋ₓMgₓ)(Si₄)O₁₀[OH]₂M⁺ⁿ_{x/n}(montmorillonite) [Formula 1]

(Al₂)(Si₄₋ₓAlₓ)O₁₀[OH]₂M⁺ⁿ_{x/n}(beidellite) [Formula 2]

(Mg₃₋ₓLiₓ)(Si₄)O₁₀[OH]₂M⁺ⁿ_{x/n}(hectorite) [Formula 3]

(Mg₃₋ₓFe⁺³ₓ)(Si₄₋₂ₓAl₂ₓ)O₁₀[OH]₂M⁺ⁿ_{x/n}(saponite) [Formula 4]

(Al_{2-x-y}Fe_{y}Mgₓ)(Si_{4-z}Al_{z})O₁₀[OH]₂M⁺ⁿ_{(x+z)/n}(illite) [Formula 5]

wherein M is an interlayer metal ion that can be more easily substituted by another cation or a cationic organic material than an intralayer metal ion (e.g., Si, Al, or Mg), x is a composition ratio of the interlayer metal ion, 0.2 to 0.7, and n is a valence.

When x is 0.2-0.7, the interlayer cation can be easily substituted by another cation or a cationic organic material.

Montmorillonite is a 2:1 layered silicate wherein an Al2O3 octahedral layer is interposed between two SiO4 tetrahedral layers to form a Si—Al—Si layered structure. When Si atoms of the layered structure are substituted by Al atoms or when Al atoms of the layered structure are substituted by metal oxide, the SiO4 tetrahedra are negatively charged. In order to satisfy a net charge balance, monovalent or divalent alkaline metal or earth metal cations exist in an interlayer space between a Si—Al—Si layer and another Si—Al—Si layer. The most fundamental chemical composition of montmorillonite is represented by Formula 6 below:

NaO_{0.33}nH₂O(Al_{1.67}Mg_{0.33})Si₄O₁₀(OH)₂ [Formula 6]

In an embodiment of the present invention, the drug in the free-base form may be paroxetine used as an antidepressant, donepezile for the treatment of Alzheimer's disease, and sibutramin used as an anti-obesity drug, which are respectively represented by Formulae 7, 8, and 9 below:

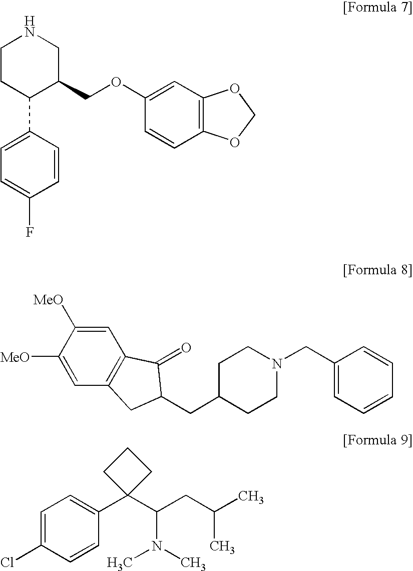

These drugs have an amine group with high basicity. The drugs are positively charged by cationic hydroxylation of the amine group, and thus, can be loaded onto inorganic carriers, such as montmorillonite, by cationic exchange between the drugs and the inorganic carriers. As the basicity of a drug increases, a cationic exchange between the drug and an inorganic carrier occurs more easily.

A reaction between a drug with high basicity and an inorganic carrier may be performed in an acidic condition of pH 1-7. As described above, this is because the drug with high basicity can be easily hydroxylated and the inorganic carrier can be easily swollen (an increase of an interlayer spacing of the inorganic carrier) in the acidic condition of pH 1-7, thereby inducing the synthesis of a drug-inorganic carrier hybrid.

Eudragit E is diversely used as an excipient or a coating agent in various formulations. Eudragit E can be selectively dissolved according to pH, and thus, is also used for selective dissolution of a drug at pH 1.2. AEA is also used for selective dissolution of a drug under an acidic condition (U.S. Pat. No. 6,056,974), for long-lasting masking of an offensive taste of a drug (Japanese Pat. No. 93-00291, U.S. Pat. No. 5,972,373), for controlling a drug dissolution (U.S. Pat. No. 4,404,183), etc.

In particular, a butylmethacrylate-(2,2-dimethylaminoethyl)methacrylate-methylmethacrylate-copolymer (Eudragit E100, Degussa) and polyvinylacetal diethylaminoacetate (AEA, Sankyo Co. Ltd) may be used herein, which are respectively represented by Formulae 10 and 11 below. As represented by Formulae 10 and 11, these polymers have a basic functional group, and can be easily substituted for a drug loaded onto an inorganic carrier by cationic hydroxylation of the basic functional group. The polymers inserted in the interlayer of the inorganic carrier can maintain a net charge balance on a negatively charged surface of the inorganic carrier, thereby effectively increasing an dissolution of the drug.

Thus, polymers (e.g., chitosan and gelatin) having a similar basic functional group to the amine group of Eudragit E and AEA can effectively increase an dissolution of a drug in a free-base form loaded onto a layered silicate.

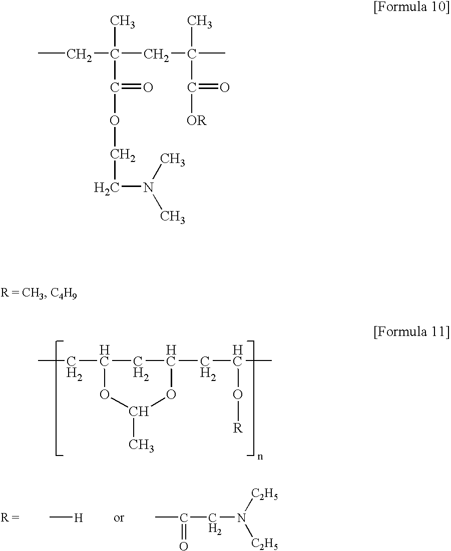

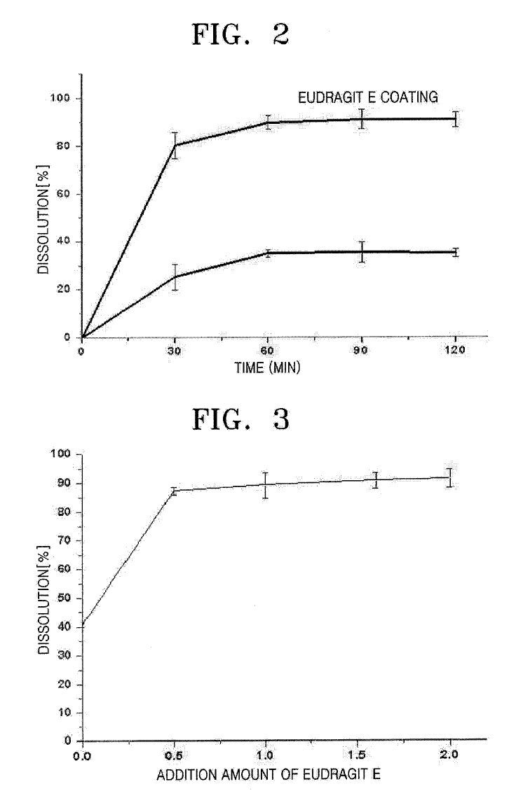

In the following working examples according to the present invention, a drug dissolution of a hybrid of montmorillonite with paroxetine, donepezile, or sibutramin was 40% or less, whereas a drug dissolution of a Eudragit E- or AEA-coated hybrid was up to 90% (see FIGS. 2, 4, and 6). In particular, Eudragit E and AEA have an inherent selective property that can be selectively dissolved in an acidic condition. Thus, a Eudragit E- or AEA-coated, drug-layered silicate hybrid according to the present invention can be effectively applied to oral formulations requiring a high drug dissolution within a short time after orally administered.

Examples of a drug that shows a high dissolution from a Eudragit E- or AEA-coated, drug-layered silicate hybrid include antibacterial agents such as ketoconazole, cefdinir, salazosulfadimidine, fluconazole, cefuroxime, cephalexin, cefadroxil, cefroxadine, formoterol, ciprofloxacin, and oflofioxacin; antiviral agents such as acyclovir and famciclovir; antibiotics such as tobramycin, cefixime, ceftriaxone, cefminox, cefetamet, pivoxil, cefuroxime, lomefloxacin, sparfloxacin, roxithromycin, cefpodoxime proxetil, and potassium clavulanate; antiallergic agents such as ketotifen; anti-inflammatory agents such as epirizole, aceclofenac, acetaminophen, diclofenac, piroxicam, tolfenamic, sulindac, oxaprozin, acetaminophen, aceclofenac, talniflumate, and meloxicam; antihistamines such as azelastine; antidepressants such as sertraline, dothiepin, buspirone, fluoxetine, risperidone, dl-methionine, and butabarbital (for the treatment of insomnia); mood stabilizers such as alprazolam; choline alphoscerate for the treatment of degenerative organic mental disorders; antiemetics such as ondancetron and domperidone; antihypertensive agents such as losartan, atorvastatin, nifedipine, betaxolol, bumetanide, doxazocin, ramipril, enalapril, isradipine, atenolol, bendroflumethiazid, benzthiazid, and bumetanide; hypoglycemics such as metformin and fenofibrate; vasodilators such as heptaminol HCl; carvedilol for the treatment of heart failure; cardiovascular drugs such as clopidogrel; thrombolytics such as cilostazole; antianemic drugs such as bromodiphenhydramin, ferric protein succinylate, and mecobalamin; anticancer agents such as prednimustine, UDCT, doxifluridine, and capecitabine; antidepilatory agents such as finasteride; antiulcer agents such as rebamipide, omeprazole, ranitidine, and aceglutamide (Al); gastric antisecretory agents such as misoprostol; gastrointestinal mobility enhancers such as levosulpiride; bethanechol for the treatment of neurogenic hypotonia; stimulant laxatives such as bisacodyl; antitussives & expectants such as cefixime, oxolamine, carboxymethylcysteine, erdosteine, and guaifenesin; ocular and nasal preparations such as diphexamide methiodide; antispasmodics such as tiropramid, trimebutine, and tiropramide; alendronate for the treatment of osteoporosis; and uterus constrictors such as misoprostol.

The drug inserted into the interlayer of the layered silicate, even though not used as an acidic salt form, has good stability and solubility in an aqueous solution and an ethanol solvent. However, in order for the hybrid of the drug with the above advantages and the layered silicate to be formulated into oral dosage forms, a sufficient amount of the drug must be released in gastrointestinal conditions within a predetermined time.

For this, the drug inserted into the interlayer of the layered silicate must be substituted by another cations or cationizable molecules, e.g., cationic inorganic materials such as pharmaceutically acceptable calcium, sodium, potassium, and ammonium ions, or ionizable basic organic materials. Thus, the drug-layered silicate hybrid of the present invention may include a cationic inorganic material or a basic organic material to control the dissolution of the drug. However, the cationic inorganic material is not preferable due to low exchange capacity with a drug inserted into the interlayer of a layered silicate.

Thus, the drug-layered silicate hybrid of the present invention is coated with a basic polymer to control the dissolution of the drug. Here, the basic polymer may be any cationic or cationizable organic material, and more preferably, a water-soluble cationic polymer or copolymer. Examples of the water-soluble cationic polymer or copolymer include aminoalkylmethacrylate copolymers such as dimethylaminoethylmethacrylate, aminoalkylmethacrylamide copolymers such as dimethylaminopropylmethacrylamide, cationic polysaccharides such as chitosan, and polyvinylacetal diethylaminoacetate.

In an embodiment of the present invention, the basic polymer capable of substituting for the drug inserted into the interlayer of the layered silicate may be AEA, Eudragit E100 (Degussa), and polydimethylaminoethylmethacrylate (PDMAEMA). Eudragit E100 is particularly preferable since it effectively increases a drug dissolution due to good exchange capacity with a drug.

The cationic polymer can be coated on the drug-layered silicate hybrid using any method well known in the art, e.g., direct coating, surface coating, or fluidized-bed coating. When a coating material has good affinity with a substrate, direct coating is preferable. At this time, it is preferable to use a spray drying method to achieve good coating uniformity. The spray drying method enables the creation of microparticles with a particle size of 100 micron or less, in addition to rapid drying.

The cationic polymer-coated, drug-layered silicate hybrid according to the present invention can be formulated into pharmaceutical forms, e.g., powders, granules, tablets, or capsules, using any method known in the pharmaceutical industry. The hybrid of the present invention can be used alone or in combination with a pharmaceutically acceptable additive, such as a carrier, an excipient, or a diluent. The hybrid of the present invention can be administered orally or parenterally, but is suitable for use as oral formulations due to good stability and dissolution. In the hybrid of the present invention, the drug in the free-base form may be used in an amount of 0.01 to 10 wt % based on the total weight of a hybrid composition.

Hereinafter, the present invention will be described more specifically by the following working examples. However, the following working examples are for illustrative purposes and are not intended to limit the scope of the present invention.

### EXAMPLE 1

5 g of montmorillonite used as a layered silicate was dispersed in 500 ml of distilled water, and a hydrochloric acid or a phosphoric acid was added to the dispersion solution so that pH of the dispersion solution was set to 3. A solution of 1.93 g of a free-base form of paroxetine in 200 ml of ethanol was added to the resultant montmorillonite-containing dispersion solution, and the solution was stirred for 3 hours. The resultant solution was filtered, washed with water, and spray-dried.

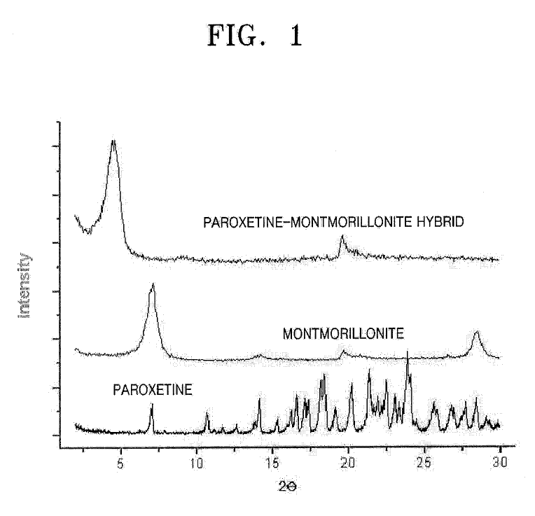

A paroxetine-montmorillonite hybrid was identified by X-ray diffraction analysis, and the X-ray diffraction pattern of the paroxetine-montmorillonite hybrid is shown in FIG. 1.

The paroxetine-montmorillonite hybrid was quantified using a UV spectroscope. As a result, the content of paroxetine was 27.65%.

### EXAMPLE 2

5 g of montmorillonite used as a layered silicate was dispersed in 500 ml of distilled water, and a hydrochloric acid or a phosphoric acid was added to the dispersion solution so that pH of the dispersion solution was set to 3. A solution of 1.93 g of a free-base form of paroxetine in 200 ml of ethanol was added to the resultant montmorillonite-containing dispersion solution, and the solution was stirred for 3 hours.

The resultant solution was filtered, washed with water, and dispersed in 250 ml of ethanol. A solution of 3.1 g of Eudragit E in 200 ml of methylenechloride was added to the dispersion solution, and the solution was stirred for one hour.

The resultant solution was spray-dried to give a Eudragit E-coated paroxetine-montmorillonite hybrid. The content of paroxetine, as determined by a UV spectroscope, was 17.23%.

### EXAMPLE 3

5 g of montmorillonite used as a layered silicate was dispersed in 500 ml of distilled water, and a hydrochloric acid or a phosphoric acid was added to the dispersion solution so that pH of the dispersion solution was set to 3. A solution of 1.93 g of a free-base form of paroxetine in 200 ml of ethanol was added to the resultant montmorillonite-containing dispersion solution, and the solution was stirred for 3 hours.

The resultant solution was filtered, washed with water, and dispersed in 250 ml of ethanol. A solution of Eudragit E (0.96 g, 1.93 g, 3.86 g, and 5.79 g, respectively corresponding to 0.5-, 1-, 1.62-, and 2-fold of the weight of paroxetine) in 200 ml of methylenechloride was added to the dispersion solution, and the reaction solutions were stirred for one hour.

The resultant solutions were spray-dried to give Eudragit E-coated paroxetine-montmorillonite hybrids. The contents of paroxetine, as determined by a UV spectroscope, were 24.15, 20.65, 17.23, and 13.73%, respectively.

### EXAMPLE 4

The dissolution tests for the paroxetine-montmorillonite hybrid prepared in Example 1 and the Eudragit E-coated paroxetine-montmorillonite hybrids prepared in Examples 2 and 3 were preformed with a pH 1.2 buffer, and the dissolution of paroxetine was analyzed from 30 minutes to 2 hours after dosing. The analysis of the dissolution of paroxetine was performed using a UV spectroscope.

The dissolution of paroxetine according to the presence or absence of Eudragit E coating is shown in FIG. 2 (Examples 1 and 2), and the dissolution of paroxetine according to the addition amount of Eudragit E is shown in FIG. 3 (Example 3).

### EXAMPLE 5

5 g of montmorillonite used as a layered silicate was dispersed in 500 ml of distilled water, and a hydrochloric acid or a phosphoric acid was added to the dispersion solution so that pH of the dispersion solution was set to 2. A solution of 4.7 g of a free-base form of donepezile in 300 ml of a mixed solvent of methylenechloride and ethanol was added to the resultant montmorillonite-containing dispersion solution, and the solution was stirred for 3 hours. The reaction solution was filtered, washed with water, and spray-dried.

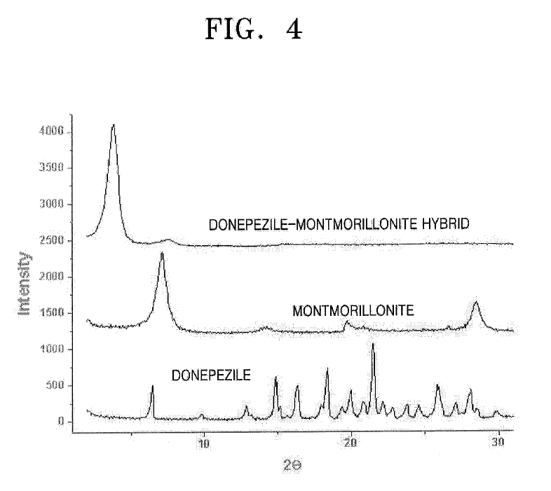

A donepezile-montmorillonite hybrid was identified by X-ray diffraction analysis, and the X-ray diffraction pattern of the donepezile-montmorillonite hybrid is shown in FIG. 4.

The donepezile-montmorillonite hybrid was quantified by high-performance liquid chromatography (HPLC). As a result, the content of donepezile was 28%.

### EXAMPLE 6

5 g of montmorillonite used as a layered silicate was dispersed in 500 ml of distilled water, and a hydrochloric acid or a phosphoric acid was added to the dispersion solution so that pH of the dispersion solution was set to 2. A solution of 4.7 g of a free-base form of donepezile in 300 ml of a mixed solvent of methylenechloride and ethanol was added to the resultant montmorillonite-containing dispersion solution, and the solution was stirred for 3 hours.

The reaction solution was filtered, washed with water, and dispersed in 250 ml of ethanol. A solution of 2.0 g of Eudragit E in 70 ml of methylenechloride was added to the dispersion solution, and the solution was stirred for one hour.

The resultant solution was spray-dried to give a Eudragit E-coated donepezile-montmorillonite hybrid. The content of donepezile, as determined by HPLC, was 24%.

### EXAMPLE 7

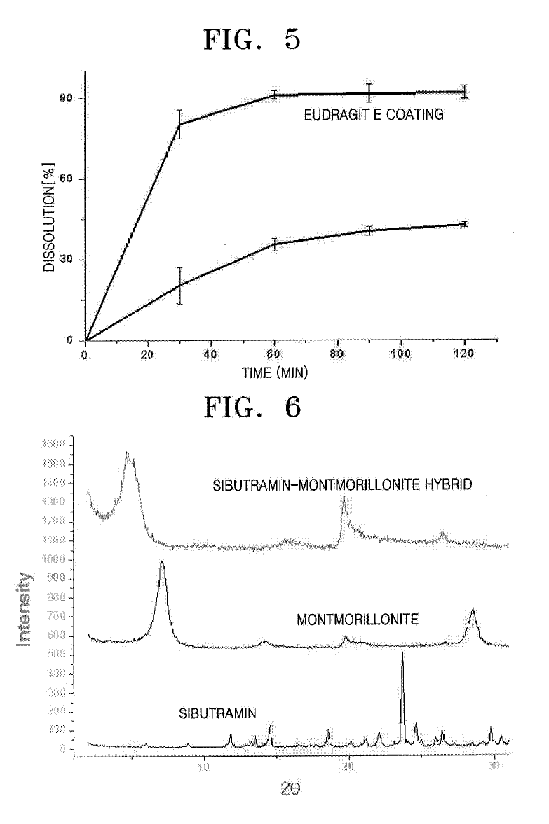

The dissolution tests for the donepezile-montmorillonite hybrid prepared in Example 5 and the Eudragit E-coated donepezile-montmorillonite hybrid prepared in Example 6 were performed with a pH 1.2 buffer, and the dissolution of donepezile was analyzed from 30 minutes to 2 hours after dosing. The analysis of the dissolution of donepezile was performed by HPLC, and the results are shown in FIG. 5.

### EXAMPLE 8

5 g of montmorillonite used as a layered silicate was dispersed in 250 ml of distilled water, and a hydrochloric acid or a phosphoric acid was added to the dispersion solution so that pH of the dispersion solution was set to 1, 3, and 6. A solution of 1.8 g of a free-base form of sibutramin in 50 ml of methanol was added to the resultant montmorillonite-containing dispersion solutions, and the solutions were stirred for 3 hours. The reaction solutions were filtered, washed with water, and spray-dried.

Sibutramin-montmorillonite hybrids were identified by X-ray diffraction analysis, and the X-ray diffraction patterns of the sibutramin-montmorillonite hybrids are shown in FIG. 6.

The sibutramin-montmorillonite hybrids were quantified using a UV spectroscope. As a result, the contents of the sibutramin-montmorillonite hybrids synthesized at pH of 1, 3, and 6 were 22.4, 24, and 21.7%, respectively.

### EXAMPLE 9

5 g of montmorillonite used as a layered silicate was dispersed in 250 ml of distilled water, and a hydrochloric acid or a phosphoric acid was added to the dispersion solution so that pH of the dispersion solution was set to 1, 3, and 6. A solution of 1.8 g of a free-base form of sibutramin in 50 ml of methanol was added to the resultant montmorillonite-containing dispersion solutions, and the solutions were stirred for 3 hours.

The reaction solutions were filtered, washed with water, and dispersed in 250 ml of ethanol. A solution of 2.5 g of Eudragit E in 100 ml of methylenechloride was added to the dispersion solutions, and the solutions were stirred for one hour.

The resultant solutions were spray-dried to give Eudragit E-coated sibutramin-montmorillonite hybrids. The contents of sibutramin in the Eudragit E-coated sibutramin-montmorillonite hybrids synthesized at pH of 1, 3, and 6, as determined by a UV spectroscope, were 19.3, 20.6, and 17.2%, respectively.

### EXAMPLE 10

5 g of montmorillonite used as a layered silicate was dispersed in 250 ml of distilled water, and a hydrochloric acid or a phosphoric acid was added to the dispersion solution so that pH of the dispersion solution was set to 1, 3, and 6. A solution of 1.8 g of a free-base form of sibutramin in 50 ml of methanol was added to the resultant montmorillonite-containing dispersion solutions, and the solutions were stirred for 3 hours.

The reaction solutions were filtered, washed with water, and dispersed in 250 ml of ethanol. A solution of 2.5 g of AEA in 100 ml of methylenechloride was added to the dispersion solutions, and the solutions were stirred for one hour.

The resultant solutions were spray-dried to give AEA-coated sibutramin-montmorillonite hybrids. The contents of sibutramin in the AEA-coated sibutramin-montmorillonite hybrids synthesized at pH of 1, 3, and 6, as determined by a UV spectroscope, were 15, 15.3, and 14.3%, respectively.

### EXAMPLE 11

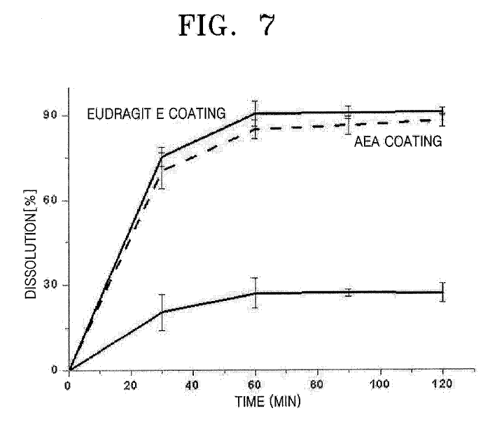

The dissolution tests for the sibutramin-montmorillonite hybrid prepared at pH of 3 in Example 8, the Eudragit E-coated sibutramin-montmorillonite hybrid prepared at pH of 3 in Example 9, and the AEA-coated sibutramin-montmorillonite hybrid prepared at pH of 3 in Example 10 were performed with a pH 1.2 buffer, and the dissolution of sibutramin was analyzed from 30 minutes to 2 hours after dosing. The analysis of the dissolution of sibutramin was performed using a UV spectroscope, and the results are shown in FIG. 7

According to the present invention, an Eudragit E- or AEA-coated, poorly soluble drug-layered silicate hybrid can increase the solubility and stability of the poorly soluble drug, and achieve an increase (up to 90%) in a short-term dissolution, and thus, is suitable for oral formulations

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A hybrid of a drug in a free-base form and a layered silicate, comprising a basic polymer capable of controlling a dissolution of the drug, wherein the drug is inserted into an interlayer of the layered silicate.

2. The hybrid of claim 1, wherein the layered silicate is selected from the group consisting of montmorillonite, beidellite, nontronite, hectorite, saponite, illite, celadonite, gluconite, clay, and bentonite.

3. The hybrid of claim 1, wherein the drug is selected from the group consisting of amlodipine, paroxetine, donepezile, and sibutramin.

4. The hybrid of claim 1, wherein the basic polymer is used in combination with an inorganic salt.

5. The hybrid of claim 1, wherein the basic polymer is a cationic polymer or copolymer.

6. The hybrid of claim 1, wherein the basic polymer is an alkylaminomethacrylate copolymer, polyvinylacetal diethylaminoacetate, or polyalkylaminoalkylmethacrylate.

7. A method of preparing the hybrid of claim 1, the method comprising:
(a) dispersing a layered silicate in an aqueous solvent to prepare a layered silicate-containing aqueous solution;
(b) dissolving a drug in a free-base form in ethanol or water to prepare a drug-containing solution;
(c) mixing the layered silicate-containing aqueous solution with the drug-containing solution while stirring to prepare a hybrid wherein the drug is inserted into an interlayer of the layered silicate; and
(d) adding a basic polymer to the hybrid.

8. The method of claim 7, wherein in (b), the ethanol or the water has pH of 1-7.

9. The method of claim 7, wherein in (d), the basic polymer is added to the hybrid using spray drying, fluidized-bed coating, vacuum drying, or common oven drying.

10. An oral formulation comprising the hybrid of claim 1 as an effective ingredient.
